(19) United States
(12) Patent Application Publication
(10) Pub. No.: US 2011/0295218 A1
(43) Pub. Date: Dec. 1, 2011

(54) BENDED INJECTION NEEDLE

(75) Inventors: Bastian Gaardsvig Kjeldsen, Copenhagen O (DK); Lasse Wengel Christoffersen, Frederikssund (DK); Kritian Glejbøl, Glostrup (DK); Salim Bouaidat, Copenhagen K (DK)

(73) Assignee: NOVO NORDISK A/S, Bagsvaerd (DK)

(21) Appl. No.: 13/128,495

(22) PCT Filed: Nov. 6, 2009
(86) PCT No.: PCT/EP2009/064763

§ 371 Date: Aug. 9, 2011

(30) Foreign Application Priority Data
Nov. 11, 2008 (EP) 08168817.8

(51) Int. Cl. A61M 5/00

(52) U.S. Cl. 604/272

(57) ABSTRACT

A method of manufacturing a bended injection needle assembly comprising the steps of: mounting a straight one-piece metallic needle cannula (20) in a hub (1), securing the straight needle cannula (20) to the hub (1) and bending the needle cannula (20) such that at least a part of the needle cannula deflects away from the first axis (X).

### THE TECHNICAL FIELD OF THE INVENTION

The invention relates to a method of producing an injection needle assembly and especially to the manufacture of bended injection needle assemblies.

### DESCRIPTION OF RELATED ART

Needle assemblies are commonly used to either inject substances into or extract substances out of human or animal bodies. Such needle assemblies are typically disposable and are discarded after only one use.

When producing such needle assemblies a needle cannula which is typically drawn from stainless steel is attached to a hub generally moulded from a suitable polymer. The hub usually has an opening stretching through the hub from a distal surface to an proximal surface. The needle cannula is typically mounted in this opening and secured to the hub by gluing or by other suitable means e.g. by welding.

Many injection devices e.g. pen systems for injecting insulin or growth hormone carry the drug in a cartridge which is sealed by a rubber membrane. In order to eject the drug from the cartridge the injection needle assembly needs to be mounted in the hub with a part of needle cannula pointing in the non-patient direction such that this non-patient part of the needle cannula can penetrate the rubber membrane. An example of such needle assembly suitable for a pen system is disclosed in WO 95/23005.

U.S. Pat. No. 5,295,827 discloses an injection needle assembly in which the part of the needle cannula that protrudes from the hub is bended such that at least the tip of the cannula is deflected away from the centre axis. This is done by inserting the cannula tip into a separate tool and manually deflecting the injection device with the needle assembly attached to an angle relatively to the tool.

WO 05/097237 discloses an injection needle assembly in which a needle cannula is first bended into a multi-angled J-shaped needle cannula which thereafter is attached to a plastic hub. This needle has a first part for penetrating into the patient and a second part for penetrating into a container carrying the drug. These two parts are parallel to each other such that the injection needle assembly in one and the same axial movement can penetrate both the patient and the container of the injection device.

When producing an injection needle with a bended needle as the one disclosed in WO 05/097237 it has proven to be very difficult to handle the bended needle cannula in a large scale production. It is especially very difficult to secure the bended needle to the hub in the correct way and with the correct alignment.

### DESCRIPTION OF THE INVENTION

It is among an object of the present invention to provide a method for manufacturing a needle assembly having a bended needle cannula and which can be more easily handled in a large scale production.

### Claim 1-4

A straight needle cannula can be handled much more easily in a production set-up than a bended needle. Most of the needle assemblies on the market today are made from straight needle cannulas which are mounted in a hub. Such straight needle cannula is usually a drawn one-piece metallic tube. This kind of production is therefore standard procedure for manufactures of needle assemblies. In the method described in this application, the needle is supported by the hub as a straight needle cannula following standard procedure. The bending itself is first done when the needle cannula is supported by the hub which means that the hub is available for holding the needle cannula during the bending process. The needle cannula need not be physically attached to the hub during the bending process but needs simply to be supported by the hub. The needle cannula can be physically attached or secured to the hub in many different ways either before bending or after bending the needle cannula.

The needle cannula may be bend in any angle but a first bend of approximately 90 degrees followed by a second bend of also approximately 90 degree is preferred such that the two ends of the needle cannula lay approximately parallel to each other i.e. a total bend of approximately 180 degrees. Also when bending the needle cannula it is preferably positioned such that the grinding angle of the two needle cannula tips are correctly located relatively to the hub.

In order for the needle cannula not to flex back due to the build-in resiliency, the bended part of the needle cannula is secured to the hub. This can be done in a number of different ways. A preferred way is to mould proper holding means into the hub, but any kind of gluing or welding could also be used.

### Claim 5-9

The hub used in the claimed method involves at least two different bending surfaces which are surfaces moulded in the hub and which are suitable to bend the needle cannula over. In this way no additional means are necessary for bending the needle cannula. Both these bending surfaces preferably facilitate a 90 degree bend of the needle cannula.

The injection needle assembly manufactured by the method described comprises a hub and a needle cannula. The hub comprises two different bending surfaces located such that the needle cannula can be bended from a straight one-piece metallic needle cannula into a configuration in which the two opposite ends of the needle cannula extends from the hub in the same parallel direction.

The hub of the needle assembly preferably has an opening for supporting the needle cannula during the bending process. The needle cannula can e.g. be secured to the hub inside this opening. Further the hub carries means for securing the bended part of the needle cannula to the hub. These means can e.g. be provided as a triangular cut-out in which a groove or trench are provided which has a longitudinal opening which is a little smaller than the diameter of the needle cannula such the needle cannula can be secured in this groove of trench.

### Definitions:

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form. Further the term “injection needle” defines a piercing member adapted to penetrate the skin of a subject for the purpose of delivering or removing a liquid.

The term “Needle Cannula” is used to describe the actual conduit performing the penetration of the skin during injection. A needle cannula is usually made from a metallic material such as stainless steel and connected to a hub to form an injection needle assembly. The “hub” being the part the needle cannula is mounted to and which carries the connecting means for connecting the needle cannula to an injection apparatus is usually moulded from a suitable thermoplastic material. The “needle assembly” is to be understood as the needle unit itself i.e. comprising a needle cannula mounted in a hub as supplied to the user.

“Cartridge” is the term used to describe the container containing the insulin. Cartridges are usually made from glass but could also be moulded from any suitable polymer. A cartridge or ampoule is preferably sealed at one end by a pierceable membrane which can be pierced e.g. by an injection needle. The opposite end is closed by a plunger or piston made from rubber or a suitable polymer. The plunger or piston can be slidable moved inside the cartridge. The space between the pierceable membrane and the movable plunger holds the insulin which is pressed out as the plunger decreased the volume of the space holding the insulin. As an alternative to a cartridge, a flexible reservoir could be used.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a perspective view of the hub.

FIG. 2 show a sectional view of the hub.

FIG. 3 show a sectional view of the needle assembly during manufacturing.

FIG. 4 show a sectional view of the needle assembly.

FIG. 5 show a perspective view of the needle assembly.

FIG. 6 show a perspective view of the needle assembly.

FIG. 7 show a perspective view of a part of the needle assembly.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

In that context it may be convenient to define that the term “distal end” in the appended figures is meant to refer to the end of the needle cannula penetrating the patient whereas the term “proximal end” is meant to refer to the opposite end of the needle cannula.

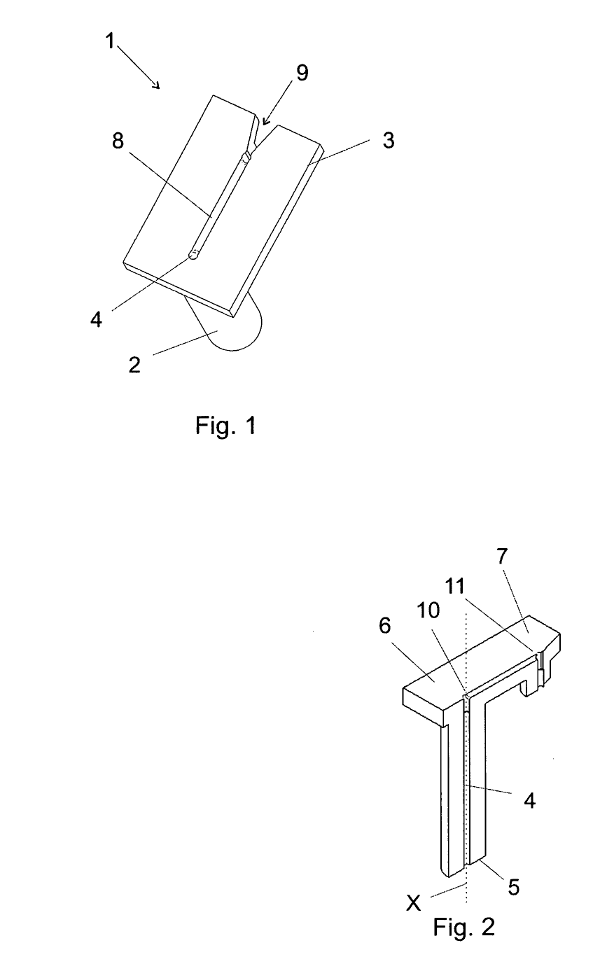

FIGS. 1 and 2 discloses a needle hub 1 comprising a first part 2 and a second part 3. The first part 2 has a circular cross section and has an opening 4 stretching through the first part 2 from its distal surface 5 to its proximal surface 6.

The second part 3 stretches in a perpendicular direction from the first part 2 and has a boxshaped configuration. The proximal surface 7 of the second part 3 is aligned with the proximal surface 6 of the first part 3 to form a joint surface 6, 7.

A channel 8 is provided in the joint proximal surface 6, 7 and terminates at the end of the second part 3 pointing away from the first part 2. At this terminating end a triangular shaped area 9 is cut-out in the second part 3.

A first bending surface 10 is provided where the opening 4 in the first part 2 connects with the channel 8 and a second bending surface 11 is provided where the channel 8 terminates in the triangular shaped cut-out 9.

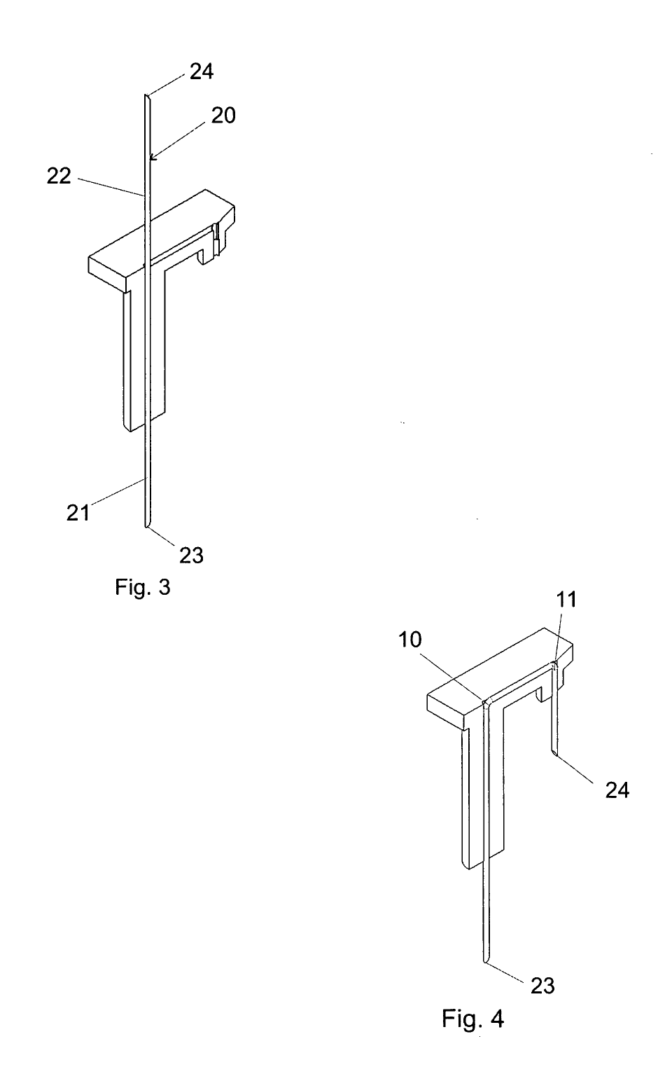

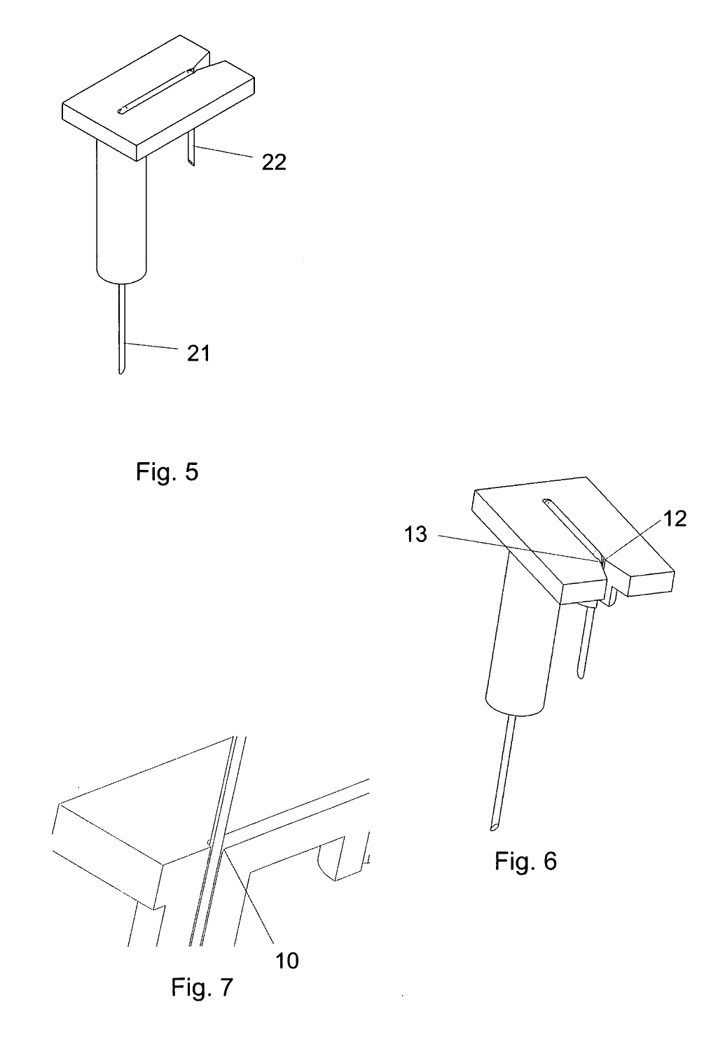

As disclosed in FIGS. 2 and 3, the needle cannula 20 is supported in a direction parallel with a first axis X. In FIG. 2 this axis X is disclosed as the centre axis of the opening 4 however an axis in any direction is within the scope of the invention.

When manufacturing the injection needle assembly a one-piece metallic tubular needle cannula 20 is first supported in the opening 4 of the first part 2 and secured to the opening 4 preferably by gluing or welding. As disclosed in FIG. 3, the needle assembly at this stage has a straight needle cannula 20 which has a distal part 21 with a first tip 23 pointing in a distal direction and a proximal part 22 with a proximal tip 24 pointing in the proximal direction.

The needle cannula 20 is hereafter bended as illustrated in FIG. 4. First over the first bending surface 10 such that the proximal part 22 of the needle cannula 20 enters the channel 8 of the second part 3, and thereafter over the second bending surface 11 such that the proximal part of the needle cannula 20 enters 2 through the triangular cut-out area 9.

The terminating end of the second part 3 of the hub 1 which terminates into the cut-out area 9 includes two ridges 12, 13 (seen in FIG. 6) which is substantially parallel with the opening 4 of the first part 2. These ridges 12, 13 locks the proximal end 22 of the needle cannula 20 as it is bended over the second bending surface 11 into a distal direction.

In use either the first tip 23 or the second tip 24 of the needle cannula 20 will, when the needle assembly is moved axially, penetrate the skin of the user while the opposite tip 23, 24 will penetrate into a not shown container such as a cartridge or a flexible reservoir. During manufacture of the needle assembly, the needle cannula 20 is oriented in the hub 1 in the way which brings the grinded angle of the tips 23, 24 in the preferred position.

Some preferred embodiments have been shown in the foregoing, but it should be stressed that the invention is not limited to these, but may be embodied in other ways within the subject matter defined in the following claims, the grinded angle could e.g. slope in any direction on the finalized needle assembly and not necessarily outwardly as disclosed in FIG. 4.

1. A method of manufacturing a bent injection needle assembly comprising:
supporting substantially straight one-piece metallic needle cannula comprising a first part with a first tip and a second part with a second tip in a hub such that the first part extends from the hub in a first direction substantially parallel to a first axis and the second part extends from the hub (1) in a second direction substantially parallel to the first axis,
securing the one-piece metallic needle cannula to the hub, and
bending the needle cannula over a first bending surface in the hub such that at least the second part deflects away from the first axis.

2. A method of manufacturing a bent injection needle assembly according to claim 1, which further comprises: bending the second part into an angle of approximately 90 degrees relative relatively to the first axis.

3. A method of manufacturing a bent injection needle assembly according to claim 2, which further comprises: bending the second part further over a second bending surface in the hub such that the second part becomes approximately parallel to the first part (21).

4. A method of manufacturing a bent injection needle assembly according to claims 1, which further comprises securing the second part of the one-piece metallic needle cannula to the hub.

5. A hub for an injection needle assembly comprising at least two different bent surfaces for a one-piece metallic needle cannula.

6. A hub according to claim 5, wherein the at least two different bent surfaces each facilitates an approximately 90 degree bend of the one-piece metallic needle cannula supported by the hub.

7. An injection needle assembly comprising:
a hub comprising at least two different bent surfaces for a one-piece metallic needle cannula, and
a one-piece needle cannula bent over the at least two different bending surfaces (10, 11) such that first part (21) and second part (22) of the one-piece needle cannula (20) is approximately parallel.

8. An injection needle assembly according to claim 7, wherein the hub comprises an opening for supporting the one-piece needle cannula.

9. An injection needle assembly according to claim 8, wherein the hub comprises means for securing the one-piece metallic needle cannula.
